(19) United States
(12) Patent Application Publication
(10) Pub. No.: US 2015/0123355 A1
(43) Pub. Date: May 7, 2015

(54) Seal Assembly Having An Anti-Friction Ring And Method Of Assembly

(71) Applicant: Teleflex Medical Incorporated, Research Triangle Park, NC (US)

(72) Inventors: Salvatore CASTRO, Raleigh, NC (US); Paul WHITING, Wakeforest, NC (US)

(21) Appl. No.: 14/535,003

(22) Filed: Nov. 6, 2014

Related U.S. Application Data
(60) Provisional application No. 61/901,174, filed on Nov. 7, 2013.

(51) Int. Cl. A61B 17/34; F16J 15/02

(52) U.S. Cl. CPC A61B 17/3417; F16J 15/022; A61B 2017/3419
USPC 277/648; 29/428

(57) ABSTRACT

A seal system may include: an annular shaped resilient seal, the resilient seal having an outer portion, an inner portion, a distal end, and a proximal end; a conical portion located in the inner portion of the resilient seal, the conical portion converging toward the distal end, the conical portion defining a hole at the distal end; an anti-friction ring; flexible fingers defining at least part of the anti-friction ring; and an end feature located at a distal part of the flexible fingers, the end feature having at least a partial spherical cross-sectional shape, wherein the anti-friction ring is dimensioned to fit, at least partially, in the conical portion of the inner portion of the resilient seal. A method of making a seal system may also be provided.

### CROSS-REFERENCE TO RELATED APPLICATIONS

This application claims the benefit of provisional application titled, SEAL ASSEMBLY HAVING AN ANTI-FRICTION RING AND METHOD OF ASSEMBLY, having application No. 61/901,174, filed on Nov. 7, 2013. The above mentioned application is incorporated by reference in its entirety.

### FIELD OF THE INVENTION

The present invention relates generally to a seal assembly having an anti-friction ring. More particularly, the present invention relates to a surgical port having a seal assembly configured to form a seal with instruments extending through the surgical port where the seal assembly includes an anti-friction ring.

### BACKGROUND OF THE INVENTION

Surgical ports are used in laparoscopic surgeries. A surgical port may include a trocar having a cannula. An obturator may be inserted through the cannula to assist in creating a hole or incision in the subject. Once the hole or incision is made in a subject, the cannula is inserted through the hole or incision into the subject. The trocar is removed and a variety of medical instruments may be inserted into the subject via the cannula. It may be desirable to form a seal around various elongated medical instruments that are inserted into the surgical port and cannula.

Resilient seals may be used for this purpose. In some instances, the resilient seals may include an orifice through which the elongated instruments are inserted. Insertion of the instrument may result in the stretching of the orifice. In some instances, moving an elongated instrument through the orifice of the seal may result in friction. The friction may result from two sources. First, the friction may result from the moving of the elongated instrument through the seal and second, the stretching of the orifice. Relatively high amounts of friction may be undesirable as the friction may be a hindrance to a surgeon moving instruments through the surgical port.

In addition to relatively high amounts of friction, another problem that may be associated with some current designs is that the insertion of the elongated instrument may damage or compromise the sealing ability of the seal. In some instances, the elongated instruments may not be aligned with the orifice. In such situations, the elongated instruments may tend to poke the resilient seal until the elongated instrument finds the orifice and moves through it. This poking or jabbing of a resilient seal may result in damage to the seal or may compromise its sealing capabilities.

Accordingly, it is desirable to provide a sealing system that can, in some embodiments, form a seal around an instrument that is inserted into a surgical port. In some embodiments, it may be desirable for a sealing system that may alleviate at least one of the conditions described above.

### SUMMARY OF THE INVENTION

The foregoing needs are met, to a great extent, by the present invention, wherein in one aspect an apparatus is provided that in some embodiments forms a seal around an instrument that is inserted into a surgical port. In some embodiments, a sealing system may protect the seal from being damaged by an end of an instrument and may reduce friction between the seal and an instrument.

In accordance with one embodiment of the present invention, A seal system may be provided. The seal system may include: an annular shaped resilient seal, the resilient seal having an outer portion, an inner portion, a distal end, and a proximal end; a conical portion located in the inner portion of the resilient seal, the conical portion converging toward the distal end, the conical portion defining a hole at the distal end; an anti-friction ring; flexible fingers defining at least part of the anti-friction ring; and an end feature located at a distal part of the flexible fingers, the end feature having at least a partial spherical cross-sectional shape, wherein the anti-friction ring is dimensioned to fit, at least partially, in the conical portion of the inner portion of the resilient seal.

In accordance with another embodiment of the present invention, a method of making a seal system may be provided. The method may include: providing an annular shaped resilient seal, the resilient seal having an outer portion, an inner portion, a distal end, and a proximal end; locating a conical portion in the inner portion of the resilient seal, the conical portion converging toward the distal end, the conical portion defining a hole at the distal end; inserting an anti-friction ring in the conical portion of the seal, the anti-friction ring having flexible fingers defining at least part of the anti-friction ring; and forming an end feature located at a distal part of the flexible fingers, the end feature having only rounded outer surfaces.

In accordance with yet another embodiment of the present invention, a seal system may be provided. The system may include: means for sealing having an outer portion, an inner portion, a distal end, and a proximal end; a conical portion located in the inner portion of the means for sealing, the conical portion converging toward the a distal end, the conical portion defining a hole at the distal end; a means for reducing friction; flexible fingers defining at least part of the means for reducing friction, the flexible fingers and dimensioned to not contact each other and have a flexible hinge portion that connects the flexible finger to the remainder of the anti-friction ring that is as wide as the width of the flexible finger adjacent to the hinge portion; and means for contacting the means for sealing located on a distal part of the flexible fingers, wherein the means for reducing friction is dimensioned to fit, at least partially, in the conical portion of the inner portion of the means for sealing.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is assembled perspective view of a surgical port or trocar in accordance with an embodiment described herein.

FIG. 2 is an exploded, perspective view of the surgical port or trocar shown in FIG. 1.

FIG. 3 is a side view of a seal used in accordance with this disclosure.

FIG. 4 is a cross-sectional view of the seal shown in FIG. 3 taken along the line 4-4.

FIG. 5 is a top view of two anti-friction rings in accordance with an embodiment of the disclosure.

FIG. 6 is a side view of the two anti-friction rings shown in FIG. 5.

FIG. 7 is a cross-sectional view of one of the anti-friction rings shown in FIGS. 5 and 6.

FIG. 8 is an enlarged, partial, detail view of the cross-sectional view taken about detail 8 in FIG. 7.

FIG. 9 is a cross-sectional view of one of the anti-friction rings shown in FIGS. 5 and 6.

FIG. 10 is an enlarged, partial, detail view of the cross-sectional view taken about detail 10 in FIG. 9.

FIG. 11 is a partial cross-sectional view of an end of an instrument entering the anti-friction ring and seal.

FIG. 12 is a partial cross-sectional view of an instrument where the end has passed through the anti-friction ring and seal and a seal is formed round the instrument and the hole in the seal.

### DETAILED DESCRIPTION

The invention will now be described with reference to the drawing figures, in which like reference numerals refer to like parts throughout. An embodiment in accordance with the present invention provides a surgical port having an internal seal system to create a seal around an elongated instrument inserted into the surgical port. The seal system includes a resilient seal and anti-friction ring. In some embodiments, the anti-friction ring helps to open an orifice associated with the resilient seal as an instrument is inserted into the surgical port so that by the time the instrument contacts the orifice, the anti-friction ring is already partially enlarged the orifice.

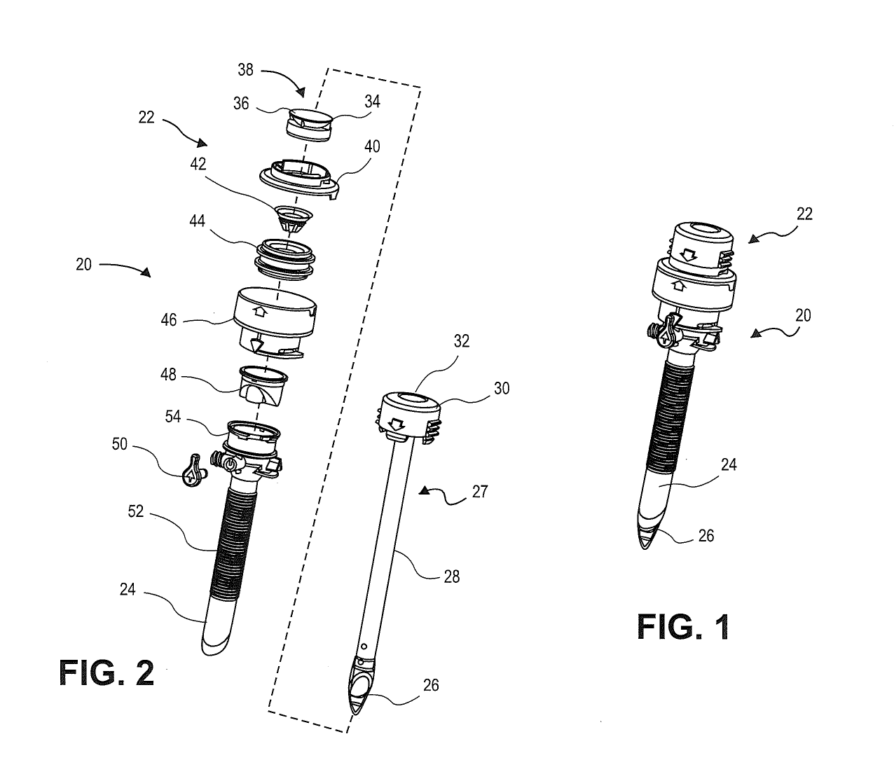

The term “distal” refers to the direction toward the bottom of the surgical port 20 as oriented in FIGS. 1 and 2. The term “proximal” refers to the direction toward the top of the surgical port 20 as oriented in FIGS. 1 and 2.

An embodiment of a surgical port 20 in accordance with the present disclosure is shown in FIGS. 1 and 2. FIG. 1 is an assembled view of the surgical port 20. FIG. 2 is an exploded view. The surgical port 20 may also be referred to as a trocar 20. The surgical port 20 includes a cap assembly 22 attached on top of a cannula 24. The cannula 24 is a long hollow member that may be inserted into a subject. The obturator 27 may be placed through the cap assembly 22 and cannula 24 such that the tip 26 extends below the cannula 24. The tip end 26 may be sharp or dimensioned so that it a can assist in cutting or moving flesh and tissue while positioning the surgical port 20.

The obturator 27 includes a shaft 28 and an obturator cap 30. The obturator cap 30 has a hole 32 so that instruments may be inserted into the obturator 27. An example of an instrument that may be inserted into the obturator cap 30 is a camera that may be extended into the tip 26 which, in some instances may be clear. This will allow the user to position the surgical port 20 in a subject at a desired location.

The cap assembly 22 includes a floating seal cap 34. The floating seal cap 34 may have a conical top 36. The conical top 36 may assist a user in placing the obturator 27 into the hole 38 in the floating seal cap 34 by having the conical portions sloped toward the hole 38 in the floating seal cap 34. The cap assembly 22 also includes a caged seal cap 40. The caged seal cap 40 helps to contain the anti-friction ring 42 and seal 44 in the seal housing 46. The seal housing 46 helps contain and/or secure the duckbill seal 48. The seal housing 46 is mounted to the cannula 24. In some embodiments in accordance with the present disclosure, the anti-friction ring 42 and the seal 44 may be contacted by an elongated instrument that is inserted into the entry port 20. For example, if the obturator 27 is inserted into the entry port 20 as contemplated in FIG. 2, the shaft 28 of the obturator 27 will contact the anti-friction ring 42. The seal 44 will form a seal between the shaft 28 and the seal 44. Further discussion regarding how this sealing will be accomplished will be provided later below.

At the proximal end of the cannula 24 is a wider portion forming a duckbill seal housing 54. The caged seal housing 46 attaches to the duckbill seal housing 54. This provides at least two advantages. First, the duckbill seal 48 is trapped between the duckbill seal housing 54 and the caged seal housing 46. Second, this connection also allows the cap assembly 22 to be connected to the cannula 24. The cannula 24 may include a stopcock handle 50 that may be rotated between an on and off or open and closed position. The cannula 24 may also include ribs 52.

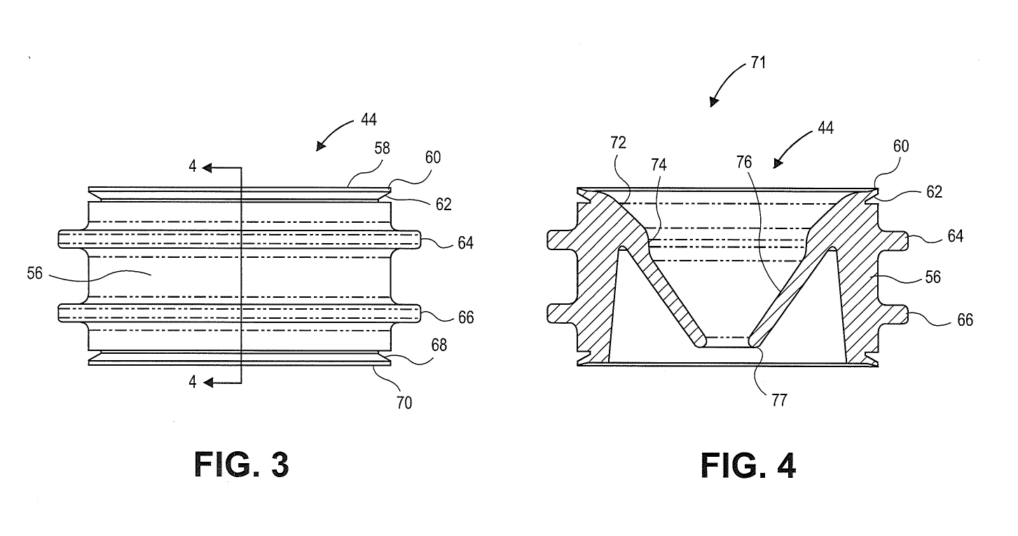

The description contained herein of the entry port/trocar 20, the cannula 24, and obturator 27 are primarily designed to provide context for the following discussion of the anti-friction ring 42, seal 44 and how they reside within the caged seal housing 46. Further detailed discussion of the entry port/trocar 20, cannula 24 and obturator 27 is not relevant and will be foregone in favor of additional description of features found in various embodiments of the anti-friction ring 42 and seal 44. This additional description will be made with reference to FIGS. 3-12.

FIG. 3 is a side view of a seal 44 in accordance of the present disclosure. FIG. 4 is a cross-sectional view of the seal shown in FIG. 3. The following discussion will be made with reference to both FIGS. 3 and 4. FIGS. 3 and 4 show a resilient seal 44. In some embodiments, the seal 44 may be referred to as a floating or caged seal 44 because it is not fixed within the seal housing 46, but rather may slide radially within the housing 46. In other embodiments, the seal 44 may be fixed within the seal housing 46. The seal 44 includes a seal body 56. The seal body 56 has a proximal end 58 which includes a proximal lip 60. In some embodiments, a groove 62 may be located adjacent to the proximal lip 60. The seal body 56 may also define one or more ribs 64 and 66. The ribs 64 and 66 may be annularly shaped and extend around the seal body 56. The seal body 56 may also include a distal groove 68 located adjacent to a distal lip 70.

In some embodiments, the proximal lip 60 and distal lip 70 may be slightly compressed thereby making the proximal groove 62 and distal groove 68 smaller when the seal 44 is located within the seal housing 46. However, in some embodiments the proximal lip 60 and the distal lip 70 are not so compressed as to eliminate the ability of the seal 44 to move radially within the seal housing 46.

As shown in FIG. 4, the seal 44 has an large diameter hole 71. The seal 44 also includes a transitional area 72 that may be tapered as shown. The seal 44 may also include a vertical wall section 74 and a conically shaped portion 76. The transitional area 72, the vertical wall section 74, and the conically shaped portion 76 may be dimensioned to receive the anti-friction ring 42. The conically shaped portion 76 may be terminated by a hole 77 (which may also be referred to as an orifice). In some embodiments, it is the conically shaped portion 76 near the hole or orifice 77 that engages an elongated tool being inserted through the entry port 20. For example, in instances where the obturator 27 is inserted into the entry port 20, the shaft 28 of the obturator 27 may be dimensioned to have a slightly larger diameter than the hole 77. As result, when the shaft 28 moves through the hole 77, the hole 77 will stretch to accommodate the outer diameter of the shaft 28. In some embodiments, the conical portion 76 near the hole 77 of the seal 44 will seal to the shaft 28 of the obturator 27. It is to be understood that while an obturator 27 has been described to be inserted into the entry port 20 and sealed to the seal 44, this is been described by way of example only. Those of ordinary skill the art reviewing this disclosure will understand that a variety of tools or instruments having various diameters can be inserted into the surgical port 20 and seal to the seal 44 in such a manner as described above with respect to the obturator 27.

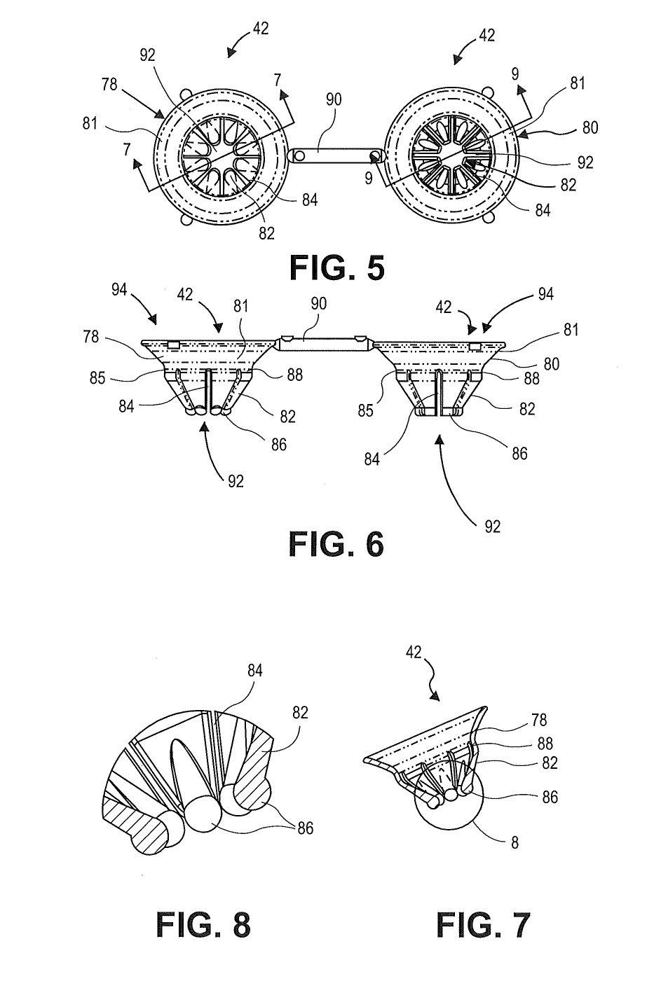

FIG. 5-10 are various views of two embodiments of anti-friction rings 42 that may be used with the seal 44 illustrated in FIGS. 2-4. FIGS. 5 and 6 are top and side views respectively. The anti-friction ring 42 on the left illustrates and a first embodiment 78 anti-friction ring 78 and the anti-friction ring 42 on the right illustrates a second embodiment 80 anti-friction ring 80. Both embodiments and of anti-friction rings 78 and 80 are similar. The similar features will be described together and the differences between the two embodiments 70 and 80 will be described separately further below.

The anti-friction rings 78, 80 include bowl shaped portions 81. The bowl shaped portion 81 has flexible fingers 82 attached to the distal portion of the bowl shaped portion 81. The fingers 82 are separated by slits 84. The bowl shaped portion 81 fits into the transition area 72 in the seal 44. The fingers 82 have straight portions 85 which fit in against the vertical wall section 74 of the seal 44. The fingers 82 are biased to a conical shape as shown in FIGS. 6-10. When set in the seal 44, the fingers 82 may rest against the conic portion 76 of the seal 44 or slightly spaced apart from the conic section 76 of the seal 44 depending upon the embodiment.

The fingers 82 are terminated at their distal ends with end features 86. Different geometry of the end features 86 are the characterizing differences between the anti-friction ring 42 of the first embodiment 78 and the anti-friction ring 42 of those second embodiment 80. The fingers 82 may attach to the bowl portion 81 of the anti-friction rings 42 via an attaching portion 88. The anti-friction rings 42 have distal openings 92 and proximal openings 94 which together form a through hole which permit an instrument to pass through the anti-friction ring 42.

In some embodiments, the anti-friction rings 42 have eight fingers 82. In other embodiments, greater or fewer fingers 82 may be used. The flexible fingers 82 are attached to the bowl portion 81 via the attaching portions 88. In some embodiments, the attaching portions 88 are approximately the same width as the portion of the fingers 82 where the fingers 82 connect to the connecting portion 88. The fingers 82 may decrease in width toward the distal end as the slits 84 converge moving towards the distal end as shown. The connecting portion 88 is separated by the slits 84 and may be considered a part of the fingers 82.

In some embodiments, the anti-friction rings 42 may be manufactured or molded together as a pair. In such embodiments, the anti-friction rings 42 may be connected to each other by a connector 90 as shown in FIGS. 5 and 6. However the connector 90 is merely an artifact of manufacturing and is removed prior to the anti-friction ring 42 being inserted into a seal 44.

FIGS. 7 and 8 are cross-sectional views of the first embodiment 78 of the anti-friction ring 42. FIG. 8 shows an enlarged section of FIG. 7. In the first embodiment 78 of the anti-friction ring 42, the attachment portion 88 is shown attaching the fingers 82 to the anti-friction ring 42. The fingers 82 are separated by slits 84. The end features 86 at the distal end of the fingers 82 are generally spherical in shape. The end features 86 comprise a shape slightly larger than a hemisphere and not quite as large as a sphere as shown. When combined with the fingers 82 the features 86 may form a full sphere. In other embodiments, other shapes for the end features 86 may be used.

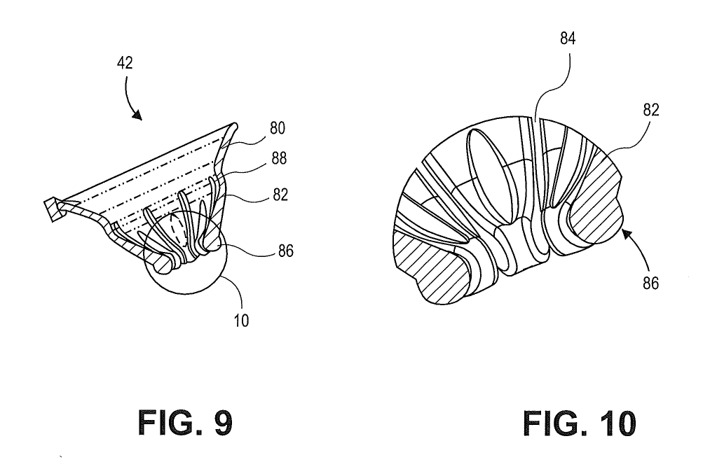

FIGS. 9 and 10 are cross-sectional views of the second embodiment 80 of the anti-friction ring 42. FIG. 10 shows an enlarged section of FIG. 9. In the second embodiment 80 of the anti-friction ring 42, the attachment portion 88 is shown attaching the fingers 82 to the anti-friction ring 42. The fingers 82 are separated by slits 84. The end features 86 at the distal end of the fingers 82 are generally spherical in shape. The end features comprise a rounded shape that, in cross-section, is similar in size to the end features 86 of embodiment one 78. However, the rounded end features 86 of embodiment two 80 is not as spherical as shown. In other embodiments, other shapes for the end features 86 may be used.

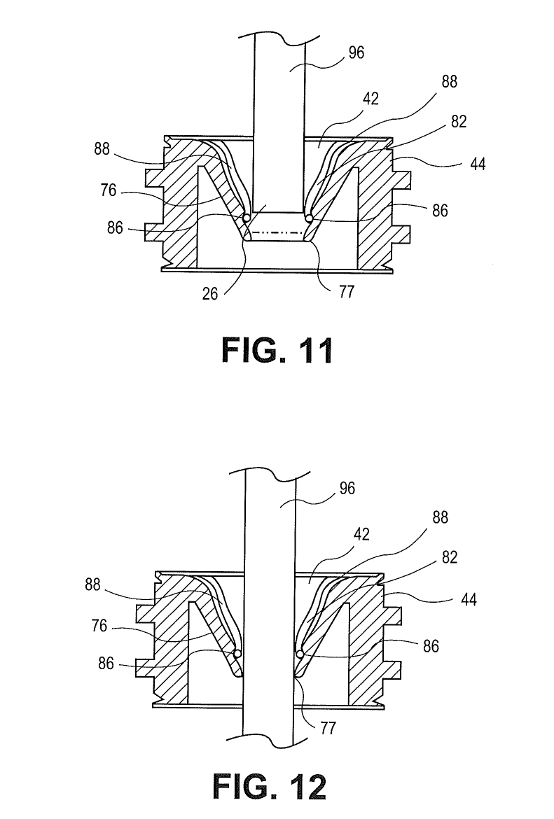

In some embodiments, an elongated instrument 96 is inserted into the assembled surgical port 20. FIGS. 11 and 12 show a seal 44 including the anti-friction ring 42. An instrument 96 is partially engaged with the anti-friction ring 42 and seal 44. The fingers 82 will flex radially outwardly to accommodate the relatively large diameter instrument 96. In some embodiments, much of the flexing of the fingers 82 will occur along the attaching portion 88. In other embodiments, flexture of the fingers 82 will occur along various locations the fingers 82 and not be confined to the attaching portion 88.

As shown in FIG. 11, the elongated instrument 96 (for example a tip 26 of the obturator 27 will initially contact the anti-friction ring 42 rather than the seal 44. This may help protect the resilient seal 44 from damage resulting from contact from the tip 26. As the tip 26 (or end of any other instrument) continues to move distally, the fingers 82 will flex radially outwardly causing the end features 86 and, in some embodiments, the actual fingers themselves 82 to contact the conical portion 76 of the seal 44. Continued distal movement of the tip 26 (or end of another instrument) will cause the end features 86 and or fingers 82 to move the conical portion 76 radially outwardly thereby enlarging the hole or orifice 77.

Enlarging the hole or orifice 77 will allow the tip 26 (or end of another instrument) to move through the hole 77 with reduced friction. In some embodiments, the hole 77, even when enlarged due to the action of the fingers 82 and or end features 86, will be dimensioned to be smaller than the diameter of the shaft 28 of the obturator 27 (or another instrument 96) so that the conical portion 76 of the seal 44 will stretch at the hole 77 to allow the instrument 96 to pass through. One of features of the stretching will be that a seal will be formed between the seal 44 and the instrument 96.

FIG. 12 shows an embodiment where the instrument 96 has fully extended through the seal 44. The fingers 82 have flexed at the attaching portions 88. In some embodiments, the fingers 82 may flex along their length. The end features 86 have engaged both the instrument 96 and the conical section 76 of the seal 44. The hole 77 has become enlarged to permit the instrument 96 to pass through the conical section 76. The hole 77 has formed a seal with the instrument 96.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A seal system comprising:
an annular shaped resilient seal, the resilient seal having an outer portion, an inner portion, a distal end, and a proximal end;
a conical portion located in the inner portion of the resilient seal, the conical portion converging toward the distal end, the conical portion defining a hole at the distal end;
an anti-friction ring;
flexible fingers defining at least part of the anti-friction ring; and
an end feature located at a distal part of the flexible fingers, the end feature having at least a partial spherical cross-sectional shape,
wherein the anti-friction ring is dimensioned to fit, at least partially, in the conical portion of the inner portion of the resilient seal.

2. The seal system of claim 1, further comprising a rib located about a circumference on the outer portion of the seal.

3. The seal system of claim 2, further comprising a second rib located about the circumference on the outer portion of the seal.

4. The seal system of claim 1, wherein the flexible fingers are configured in a generally conical configuration.

5. The seal system of claim 4, wherein the flexible fingers are attached to the rest of the anti-friction ring by a hinge portion having about the same width as a portion of the flexible finger adjacent to the hinge portion.

6. The seal system of claim 1, wherein the flexible fingers do not contact each other.

7. The seal system of claim 1, wherein the anti-friction ring has eight flexible fingers.

8. The seal system of claim 1, further comprising a second conical section in the inner portion of the seal located proximally from the conical section that defines a hole.

9. The seal system of claim 8, further comprising a cylindrical section in the inner portion of the seal distal from the second conical section and proximal from the conical section that defines the hole.

10. The seal system of claim 1, wherein the flexable fingers are configured to flex as an instrument is inserted into the anti-friction ring.

11. The seal system of claim 10, wherein the end features located on the flexable fingers urge against the conical portion of the seal when an instrument is inserted into the anti-friction ring to enlarge the hole.

12. The seal system of claim 1, further including a surgical port having a housing in which the seal system is located.

13. The seal system of claim 12, wherein the seal system is free to move radially within the housing.

14. The seal system of claim 1, wherein the partial spherical cross-sectional shape is at least half a spherical shape.

15. A method of making a seal system comprising:
providing an annular shaped resilient seal, the resilient seal having an outer portion, an inner portion, a distal end, and a proximal end;
locating a conical portion in the inner portion of the resilient seal, the conical portion converging toward the distal end, the conical portion defining a hole at the distal end;
inserting an anti-friction ring in the conical portion of the seal, the anti-friction ring having flexible fingers defining at least part of the anti-friction ring; and
forming an end feature located at a distal part of the flexible fingers, the end feature having only rounded outer surfaces.

16. The method of claim 15, wherein there are eight flexible fingers.

17. The method of claim 15, further comprising dimensioning the flexible fingers to not contact each other and have a flexible hinge portion that connects the flexible finger to the remainder of the anti-friction ring that is as wide as the width of the flexible finger adjacent to the hinge portion.

18. A seal system comprising:
means for sealing having an outer portion, an inner portion, a distal end, and a proximal end;
a conical portion located in the inner portion of the means for sealing, the conical portion converging toward the a distal end, the conical portion defining a hole at the distal end;
a means for reducing friction;
flexible fingers defining at least part of the means for reducing friction, the flexible fingers and dimensioned to not contact each other and have a flexible hinge portion that connects the flexible finger to the remainder of the anti-friction ring that is as wide as the width of the flexible finger adjacent to the hinge portion; and
means for contacting the means for sealing located on a distal part of the flexible fingers,
wherein the means for reducing friction is dimensioned to fit, at least partially, in the conical portion of the inner portion of the means for sealing.

19. The seal system of claim 18, further including a surgical port having a housing in which the seal system is located, wherein the seal system is free to move radially within the housing.

20. The seal system of claim 19, wherein the means for contacting has, at least in part, a spherical outer surface.
